(19) United States
(12) Patent Application Publication
(10) Pub. No.: US 2005/0282793 A1
(43) Pub. Date: Dec. 22, 2005

(54) Steroid prodrugs with androgenic action

(76) Inventors: Ralf Wyrwa, Rothenstein (DE); Sven Ring, Jena (DE); Peter Droescher, Weimar (DE); Alexander Hillisch, Jena (DE); Walter Elger, Berlin (DE); Birgitt Schneider, Jena (DE); Gudrun Reddersen, Jena (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/134,617

(22) Filed: May 23, 2005

Related U.S. Application Data
(60) Provisional application No. 60/572,873, filed on May 21, 2004.

(51) Int. Cl. A61K 31/56; C07J 1/00

(52) U.S. Cl. 514/182; 552/626

(57) ABSTRACT

This invention relates to steroid prodrugs with androgenic action of general formula (I), in which group Z is bonded to the steroid, 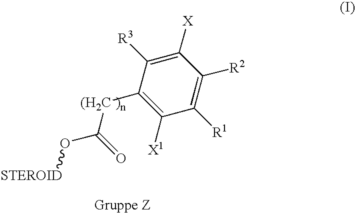 pharmaceutical compositions that contain these compounds as well as their use for the production of pharmaceutical agents with androgenic action.

This application claims the benefit of the filing date of U.S. Provisional Application Ser. No. 60/572,873 filed May 21, 2004.

The invention relates to steroid prodrugs of general formula (I), 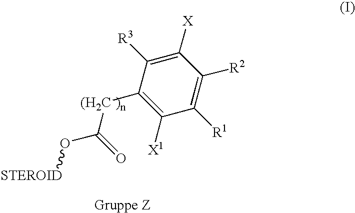
process for their production, pharmaceutical compositions that contain these compounds as well as their use for the production of pharmaceutical agents with androgenic action.

Androgens play an important role in the organism in both sexes. They exert their action via a nuclear receptor, the androgen receptor. The latter plays a role in many organs and tissues. Androgens are essential for the male reproductive function, but this constitutes only a small segment of their role in the organism. The latter comprises many metabolic functions, manifestation and preservation of the skeletal system and the muscles, functions of the liver and kidneys, the central nervous system (CNS) and the skin, and still others (1).

Androgens modulate a number of functions of other hormones, in particular those of estrogens. Examples of this are estrogen actions in the uterus and the vagina that are antagonized by androgens or are modulated in an organ-specific manner. Estrogen/androgen interactions are also of great importance on the hypophyseal level and in the liver.

The hormonal properties of androgens (testosterone) can be enhanced, reduced or qualitatively altered in many tissues by enzymatic conversions^{(2).}

By the aromatization of testosterone, estradiol can be produced in target organs. The latter may very strongly overlay the action of testosterone in the central nervous system. In the prostate, the skin and the cutaneous pictures, a conversion of testosterone into 5α-dihydrotestosterone (5α-DHT) takes place. This androgen is significantly more potent than testosterone itself. The inhibition of the conversion of testosterone into 5α-DHT very greatly reduces its androgenic action. A 5β-reduction results in non-androgenic metabolites.

Androgens (testosterone) are already formed from fetal testes. This secretion plays an important role in the build-up of male genital tracts and outer sex features with a male focus. Also, in the phase of intrauterine development, the central nervous system experiences a male imprinting. Also in this process, the prenatal sexual differentiation by the androgen secretion of the fetal testes should play an important role. A female differentiation of all structures and functions first used bisexually is carried out if no androgens are present or do not go into action in the case of errors or a genetic defect of the androgen receptor⁽³⁾.

After birth, the blood levels of testosterone in boys drop to very low values to then increase again to high values during puberty. In this phase, they act in the testes on the germinal epithelium in a synergism with the gonadotropic hormones. The sex organs that are prepared under the influence of testosterone in the fetal phase are also readily dependent in later life for their function of androgens. This also applies, however, for a broad spectrum of other organs, tissues and functions, which are controlled by androgens in the two sexes or are at least androgen-sensitive⁽⁴⁾.

The skin, hair and various appendages of the skin, such as sebaceous glands and sweat glands, exhibit the visual manifestations of this. Individuals whose androgen receptor is non-functioning due to a genetic defect exhibit typical sexual differentiation disorders (syndrome of the thus mentioned “testicular feminization”) and develop, based on the extent of the defect, little or no axillary hair and pubic hair⁽⁵⁾. Their anabolic effects and other actions on the metabolism that relate to the entire organism are another example of a role of the androgens outside of the immediate reproductive functions. The heavier muscles in males and the different accumulation and distribution of fatty tissue in males and females are expression and example of corresponding androgenic actions. Androgens stimulate the secretion of IGF-1, the most important somatotropic factor in the liver. The formation of new red blood corpuscles is also positively influenced by the mediation of the hormone erythropoietin⁽⁶⁾. Androgens suppress the growth of mammary glands in males.

In females, beginning with puberty, androgens are also secreted in physiologically relevant amounts. The adrenal glands, with androstenedione and dehydroepiandrosterone, secrete two steroids that can be converted in organs with corresponding enzyme makeup into testosterone and 5α-dihydrotestosterone⁽⁷⁾. This conversion should play a major role especially in the skin. The ovaries secrete androgen, mainly testosterone. Its secretion increases significantly mid-cycle to decrease again in the luteal phase. There can be no doubt that the libido in the two sexes is influenced positively by androgens⁽⁸⁾. For the condition, as has been proved, adrenal androgens play a major role. In women who are treated with glucocorticoids, the entire adrenal hormone production is suppressed. Depressive mood disorders that can be significantly improved by a substitution treatment with dehydroepiandrosterone are associated therewith⁽⁹⁾.

At older ages, the testosterone levels in males drop significantly on average. In this connection, a large individual variation consists of normal to excessive hypogonadal values even in the oldest ages. Still more pronounced is the loss of androgens as women age. An important source of androgens dries up with the elimination of maturing follicles. Later, the androgen secretion of the ovarian stroma also dries up. With adrenopause the adrenal androgen secretion also drops to very low values several years after the onset of menopause.

In the two sexes, at older ages, but also even before under special conditions, androgen levels that are too low and their consequences for ill-health, libido and metabolic functions can occur. In this connection, in particular somatotropic functions that are manifested by loss of muscle and bone mass and anemia are affected⁽¹⁰⁾. The consequences of androgen levels that are too low also include, however, depressive mood disorders, libido disorders and low drive.

In the oral treatment of the above-mentioned disorder pictures, testosterone is well-tolerated by the human liver but must be used in extremely high dosages to achieve therapeutically relevant blood levels. Methyl testosterone and other derivatives have a better oral activity, but are associated with the problem of poor liver compatibility⁽¹¹⁾.

Steroidally active compounds that are bonded to erythrocytes via the group —SO₂NR¹R² and accumulate there are known from DE 100 27 887.6 A1. The concentration ratio of the compounds between erythrocytes and plasma is 10-1000, preferably 30-1000, so that it is possible to speak of a depot formation in the erythrocytes. By the strong binding of the compounds to the erythrocytes, the metabolization while passing through the liver is avoided. Disadvantageously, despite a reduced metabolism with the indicated dosages, no therapy-relevant active ingredient levels are provided.

It is the object of the invention to prepare new steroidal compounds with androgenic action that are orally available and, in comparison to the prior art, also ensure a therapy-relevant active ingredient level even at a lower dosage.

This object is achieved by steroid prodrugs of general formula (I), in which a group Z is bonded to the steroid that is to be released, 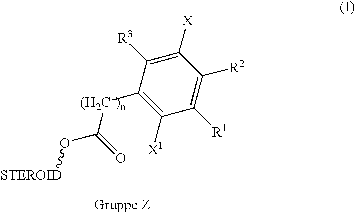
in which n is a number 0-4,
R¹ is a radical —SO₂NH₂ or —NHSO₂NH₂,
   whereby R², R³ and X, X¹ stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰, or OC(O)NH—R²¹,
   whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and R²⁰ in addition can mean a hydrogen, or
R² is a radical —SO₂NH₂ or —NHSO₂NH₂,
   whereby R², R³ and X, X¹ stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
   whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and R²⁰ in addition can mean a hydrogen, or
R³ is a radical —SO₂NH₂ or —NHSO₂NH₂,
   whereby R², R³ and X, X¹ stand for a hydrogen atom, a halogen atom, a nitrile group, a nitro group, a C₁₋₅-alkyl group, a
   CₚF₂ₚ₊₁ group with p=1-3, a group OC(O)—R²⁰, COOR²⁰, OR²⁰, C(O)NHR²⁰, or OC(O)NH—R²¹,
   whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and R²⁰ in addition can mean a hydrogen, and
STEROID stands for a steroidal ring system according to general formulas (AII-CII): 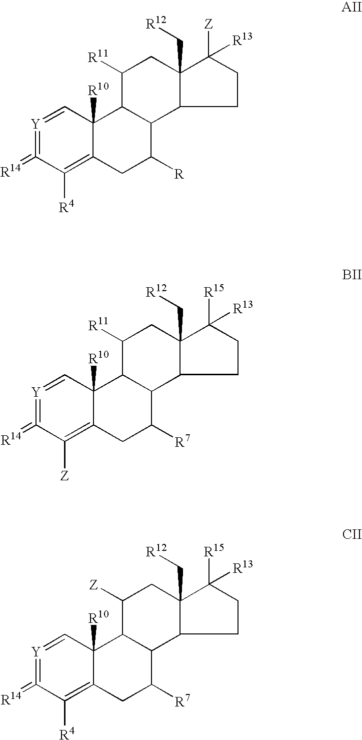
   whereby
Y represents an oxygen atom or a carbon atom,
R⁴ represents a hydrogen atom, a halogen atom, a methyl, trifluoromethyl, hydroxy, tri(C₁₋₆-alkyl)silyloxy, C₁₋₅-alkoxy or a C₂₋₅-heterocycloalkyloxy group,
R⁷ represents a hydrogen atom, a methyl or ethyl group,
R¹⁰ represents a hydrogen atom, a methyl or ethyl group,
R¹¹ represents a halogen atom, a hydrogen atom, a hydroxy group, a methoxy group, a group OC(O)—R²⁰, a methyl or ethyl group,
R¹² represents a hydrogen atom, a methyl group or an ethyl group,
R¹³ represents a hydrogen atom, a methyl, ethyl, ethinyl, trifluoromethyl, or pentafluoroethyl group,
R¹⁴ represents a hydrogen atom, an OH group, or an oxygen atom that is bonded via a double bond,
R¹⁵ represents a hydroxy, tri(C₁₋₆-alkyl)silyloxy, or C₁₋₅-alkoxy group, a group OC(O)—R²⁰ or a C₂₋₅-heterocycloalkyloxy group,
and their pharmaceutically acceptable salts.

If Y stands for a carbon atom, additional double bonds can be found in 1,2-positions, or, if radical R¹⁴ is a hydrogen atom or an OH group, in 2,3-positions. An additional double bond can also be found in 4,5-positions.

In the context of this invention, “C₁₋₅-alkyl group” is defined as a branched or straight-chain alkyl radical with 1 to 5 carbon atoms, which can be substituted by, for example, halogen atoms, hydroxy groups, or nitrile groups. As examples, a methyl, ethyl, n-propyl, i-propyl, n-butyl, i-butyl, tert-butyl or n-pentyl group can be mentioned.

The term C₁₋₅-alkoxy group is defined as a branched or straight-chain alkoxy radical with 1-5 carbon atoms. As examples, a methoxy, ethoxy, n-propoxy, i-propoxy, n-butoxy, i-butoxy, tert.-butoxy or n-pentoxy group can be mentioned.

The term tri(C₁₋₆-alkyl)silyloxy group is defined as, for example, a trimethylsilyloxy, a triisopropylsilyloxy, a thexyldimethylsilyloxy or a tert.-butyldimethylsilyloxy group.

According to the invention, the above-mentioned “C₃₋₈-cycloalkyl group” means a monocyclic or bicyclic group, which can be substituted by, for example, halogen atoms, hydroxy groups, or nitrile groups, such as, for example, a cyclopropyl, cyclobutyl, cyclopentyl, cyclohexyl or a hydroxycyclohexyl group.

In the context of this application, the term “aryl group” is defined as a substituted or unsubstituted aryl radical with 6 to 15 carbon atoms, for example, a phenyl group, a substituted phenyl group, such as a halophenyl group, or a nitrophenyl group, a diphenyl group or a naphthyl group.

In the context of this application, the term “C₁₋₄-alkylene aryl group” is defined as a disubstituted alkyl radical that is substituted at least with an aryl radical. Both radicals together have 7 to 15 carbon atoms, whereby the group can carry additional substituents, such as, for example, a halogen atom. Examples are a benzyl group or a halobenzyl group.

In the context of this application, the term “C₁₋₄-alkylene-C₃₋₈-cycloalkyl group” is defined as a disubstituted alkyl radical that is substituted at least with a C₃₋₈-cycloalkyl radical. Both radicals together exhibit 7 to 15 carbon atoms, whereby the group can carry additional substituents, such as, for example, a halogen atom. Examples are a cyclopentylethyl, cyclohexylmethyl or cyclohexylethyl group.

In the context of this application, the term “C₃₋₈-cycloalkylene-C₁₋₄-alkyl group” is defined as a disubstituted C₃₋₈-cycloalkylene radical that is substituted at least with a C₁₋₄-alkyl radical. Both radicals together exhibit 7 to 15 carbon atoms, whereby the group can carry additional substituents, such as, for example, a halogen atom. Examples are a propylcyclohexyl group or a butylcyclohexyl group.

According to this invention, the term “CₚF₂ₚ₊₁-group” is defined as a perfluorinated alkyl radical, such as, for example, a trifluoromethyl radical and a pentafluoroethyl radical.

Within the scope of the invention, the term “C₂₋₅-heterocycloalkyloxy group” is defined as a C₂₋₅-heterocycloalkyloxy group with a nitrogen atom or an oxygen atom as a heteroatom, whereby the binding of the C₁₋₅-heterocycloalkyloxy group is carried out via the oxygen atom in 2-, 3- or 4-position. An example of this is the perhydropyranoxy group.

Within the scope of this invention, the term “halogen atom” is defined as a fluorine, chlorine, bromine or iodine atom; a fluorine, chlorine or bromine atom is preferred.

The number “n” is preferably 0, 1 and 2, and especially preferably 0.

The group Z is preferably arranged in 17- and 4-position, whereby the 17-position is especially preferred.

It is preferred that R¹ represent the radical —SO₂NH₂ or —NHSO₂NH₂, whereby the radical —SO₂NH₂ is especially preferred. The above-mentioned radicals thus are found in m-position of group Z in relation to the ester group, via which group Z is bonded to the steroid.

R¹ preferably means a group —SO₂NH₂, whereby R², R³, X¹ and X preferably in each case are a hydrogen, fluorine or chlorine atom, or a hydroxy group or a methoxy group, or
R² preferably means a group —SO₂NH₂, whereby R¹, R³, X¹ and X preferably in each case are a hydrogen, fluorine or chlorine atom, or a hydroxy or a methoxy group, or
R³ preferably means a group —SO₂NH₂, whereby R¹, R², X₁ and X preferably in each case are a hydrogen, fluorine or chlorine atom, or a hydroxy or methoxy group.
Y is preferably a carbon atom.
R⁴ is preferably a hydrogen atom, a chlorine atom, or a hydroxy group.
R⁷ is preferably a hydrogen atom or a methyl group.
R¹⁰ is preferably a hydrogen atom or a methyl group.
R¹¹ is preferably a hydrogen atom, a fluorine atom or a methyl group.

R¹² is preferably a hydrogen atom.

R¹³ is preferably a hydrogen atom or a methyl group.

R¹⁴ is preferably an oxygen atom.

R¹⁵ is preferably a hydroxy group or a group OC(O)—R²⁰.

Radicals Z in 4-, 11- or 17-position, R⁴ in 4-position, R⁷ in 7-position, R¹¹ in 11-position, R¹³ in 17-position, as a function of Z or R¹⁵, and R¹⁵ in 17-position can be arranged both in α-position and in β-position.

Especially preferred steroid prodrugs are cited below:
1) 3-Oxo-7α-methylestr-4-en-17β-yl 3′-sulfamoyl benzoate (12),
2) 3-Oxo-7α-methylestr-4-en-17β-yl 4′-sulfamoyl benzoate (11),
3) 3-Oxo-7α-methylestr-4-en-17β-yl 2′-chloro-5′-sulfamoyl benzoate (13),
4) 3-Oxo-7α-methylestr-4-en-17β-yl 2′,4′-dichloro-5′-sulfamoyl benzoate,
5) 3-Oxo-7α-methylestr-4-en-17β-yl 2′-methoxy-5′-sulfamoyl benzoate,
6) 3-Oxo-7α-methylestr-4-en-17β-yl 2′,3′-dimethoxy-5′-sulfamoyl benzoate,
7) 3-Oxoestr-4-en-17β-yl 3′-sulfamoyl benzoate (7),
8) 3-Oxoestr-4-en-17β-yl 4′-sulfamoyl benzoate (8),
9) 3-Oxoestr-4-en-17β-yl 2′,4′-dichloro-5′-sulfamoyl benzoate,
10) 3-Oxoestr-4-en-17β-yl 2′-chloro-5′-sulfamoyl benzoate,
11) 3-Oxoestr-4-en-17β-yl 2′-methoxy-5′-sulfamoyl benzoate,
12) 3-Oxoestr-4-en-17β-yl 2′,3′-dimethoxy-5′-sulfamoyl benzoate,
13) 3-Oxo-7α-methylandrost-4-en-17β-yl 3′-sulfamoyl benzoate,
14) 3-Oxo-7α-methylandrost-4-en-17β-yl 4′-sulfamoyl benzoate,
15) 3-Oxo-7α-methylandrost-4-en-17β-yl 2′-chloro-5′-sulfamoyl benzoate,
16) 3-Oxo-7α-methylandrost-4-en-17β-yl 2′,4′-dichloro-5′-sulfamoyl benzoate,
17) 3-Oxo-7α-methylandrost-4-en-17β-yl 2′-methoxy-5′-sulfamoyl benzoate,
18) 3-Oxo-7α-methylandrost-4-en-17β-yl 2′,3′-dimethoxy-5′-sulfamoyl benzoate,
19) 3-Oxoandrost-4-en-17β-yl 3′-sulfamoyl benzoate (5)
20) 3-Oxoandrost-4-en-17β-yl 4′-sulfamoyl benzoate (1),
21) 3-Oxoandrost-4-en-17β-yl 2′,4′-dichloro-5′-sulfamoyl benzoate,
22) 3-Oxoandrost-4-en-17β-yl 2′-chloro-5′-sulfamoyl benzoate,
23) 3-Oxoandrost-4-en-17β-yl 2′-methoxy-5′-sulfamoyl benzoate,
24) 3-Oxoandrost-4-en-17β-yl 2′,3′-dimethoxy-5′-sulfamoyl benzoate,
25) 3-Oxoandrostan-17β-yl 3′-sulfamoyl benzoate (9),
26) 3-Oxoandrostan-17β-yl 4′-sulfamoyl benzoate (10),
27) 3-Oxoandrost-4-en-17β-yl 2′,4′-dichloro-5′-sulfamoyl benzoate (2)
28) 3-Oxoandrost-4-en-17β-yl 2′-chloro-5′-sulfamoyl benzoate (3)
29) 3-Oxoandrost-4-en-17β-yl 2′-methoxy-5′-sulfamoyl benzoate (4),
30) 3-Oxoandrost-4-en-17β-yl 2′,3′-dimethoxy-5′-sulfamoyl benzoate,
31) 3-Oxo-4-chloro-17α-methylandrosta-1,4-dien-17β-yl 3′-sulfamoyl benzoate (16),
32) 3-Oxo-4-chloro-17α-methylandrosta-1,4-dien-17β-yl 4′-sulfamoyl benzoate,
33) Androst-2-en-17β-yl 3′-sulfamoyl benzoate,
34) 3-Oxoandrost-4-en-17β-yl 2′-sulfamoyl benzoate (6),
35) Androst-2-en-17β-yl 4′-sulfamoyl benzoate,
36) 3-Oxo-7α-methyl-11β-fluoroestr-4-en-17β-yl 3′-sulfamoyl benzoate (17),
37) 3-Oxoandrost-4-en-17β-yl 4′-sulfamoylphenylpropionate (14),
38) 3-Oxo-5α-androst-1-en-17β-yl 3′-sulfamoyl benzoate (15),
39) 3-Oxoandrost-4-en-17β-yl 2′-hydroxy-5′-sulfamoyl benzoate (18),
40) 3-Oxoandrostan-17β-yl 2′-hydroxy-5′-sulfamoyl benzoate,
41) 3-Oxo-7α-methyl-11β-fluoroestr-4-en-17β-yl 2′-hydroxy-5′-sulfamoyl benzoate,
42) 3-Oxoestr-4-en-17β-yl 2′-hydroxy-5′-sulfamoyl benzoate,
43) 3-Oxo-7α-methylestr-4-en-17β-yl 2′-hydroxy-5′-sulfamoyl benzoate,
44) 3-Oxo-4-chloroandrost-4-en-17β-yl 3′-sulfamoyl benzoate (19),
45) 3-Oxo-4-chloroandrosta-1,4-dien-17β-yl 3′-sulfamoyl benzoate (20),
46) 3-Oxo-4-hydroxyestr-4-en-17β-yl 3′-sulfamoyl benzoate,
47) 3-Oxo-4-hydroxyandrost-4-en-17β-yl 3′-sulfamoyl benzoate,
48) 3-Oxo-17β-[(perhydropyran-2-yl)oxy]estr-4-en-4-yl 3′-sulfamoyl benzoate (22),
49) 3-Oxo-17β-hydroxyestr-4-en-4-yl 3′-sulfamoyl benzoate (21),
50) 3-Oxoandrost-4-en-17β-yl 2′-chloro-4′-sulfamoylbenzoate (23),
51) 3-Oxoandrost-4-en-17β-yl 3′-carboxy-5′-sulfamoylbenzoate (24),
52) 3-Oxoandrost-4-en-17β-yl 3′-carbamido-5′-sulfamoylbenzoate (25).

For the formation of pharmaceutically acceptable salts of the compounds of general formula (I) according to the invention, i.a., hydrochloric acid, hydrobromic acid, sulfuric acid and phosphoric acid are considered as inorganic acids, and, i.a., acetic acid, propionic acid, maleic acid, fumaric acid, succinic acid, benzoic acid, ascorbic acid, oxalic acid, salicylic acid, tartaric acid, citric acid, lactic acid, malic acid, mandelic acid, cinnamic acid and methanesulfonic acid are considered as organic acids.

The compounds according to the invention have androgenic activity, whereby the therapeutically relevant steroids are released by ester cleavage of the corresponding compound according to formula (I). Thus, predominantly androgens with an OH group that have a higher androgenic activity than the compounds with a keto group are advantageously released.

The oral androgenic and antigonadotropic activity as well as the influence of the liver function of the compounds according to the invention in relation to the known compounds was determined by tests on
I Sexually immature, castrated male rats,
II Sexually mature, castrated rats, and
III Gonadally intact, adult, sexually mature rats.

The results are shown graphically in FIGS. 1 to 11. Here:

FIG. 1 shows the growth of the accessory sex glands (prostate), Hershberger Test/Test 1,

FIG. 2 shows the rLH content in the serum/Test I,

FIG. 3 shows the HDL-cholesterol content in the plasma/Test I,

FIG. 4 shows the growth of the accessory sex glands (prostate), Hershberger Test/Test II,

FIG. 5 shows the rLH content in the serum/Test II,

FIG. 6 shows the HDL cholesterol content in the plasma/Test II,

FIG. 7 shows the MENT content in the serum/Test II,

FIG. 8 shows the growth of the accessory sex glands (prostate), Hershberger Test/Test III,

FIG. 9 shows the rLH content in the serum/Test III,

FIG. 10 shows the testosterone content in the serum/Test III and

FIG. 11 shows the HDL-cholesterol content in the serum/test III.

Test (I) on Sexually Immature, Castrated Male Rats Over 8 Days

Surprisingly and unexpectedly, compounds 12 or 18 according to the invention in a Test (I) on sexually immature, castrated male rats show strong dose-dependent effects on the growth of the accessory sex glands, while for (7α-methyl-19-nortestosterone) MENT, only marginal effects were observed. Unexpectedly, the substances according to the invention also show an antigonadotropic activity that is superior to that of MENT. Relative to the reduction of HDL, however, MENT is even more active than the substances according to the invention.

Description of the Test

Method: Male rats weighing about 45 g of body weight are castrated and treated orally over 7 days with MENT and with compounds 12 or 18 according to the invention. On the 8^{th} day of the test, the animals are sacrificed, and the hormonal effects of the test substances are evaluated (growth of the accessory sex glands, reduction of gonadotropic hormones in plasma, parameters of hepatic actions: effects on HDL lipoproteins).

Test (II) on Sexually Mature, Castrated Male Rats Over 8 Days

Test (II) was performed on sexually mature, castrated male rats. Also here, unexpectedly strong, dose-dependent effects on the growth of the accessory sex glands could be found with the substances according to the invention, such as, e.g., compound 12, while for MENT, only marginal effects were observed. Surprisingly enough, the compounds according to the invention also show here an antigonadotropic activity that is superior to that of MENT. Relative to the reduction of HDL, however, MENT is even more active than the substances according to the invention in this Test (II). After the oral administration of compounds according to the invention, such as, e.g., of compound 12, significantly higher levels of MENT are achieved in the plasma than after the administration of MENT itself.

Description of the Test

Method: Adult male rats, of about 300 g of body weight, are castrated and treated orally over 7 days with MENT and compound 12 according to the invention. On the 8^{th} day of the test, the animals are sacrificed, and the hormonal effects of the test substances are evaluated (growth of the accessory sex glands, reduction of the gonadotropic hormones in plasma, parameters of hepatic actions: effects on HDL lipoproteins, determination of MENT in the plasma).
   Test (III) on Gonadally Intact Adult Sexually Mature Rats Over 6 Weeks

In the third Test (III) on gonadally intact, adult, sexually mature rats over 6 weeks, all test substances had only marginal effects on the accessory sex glands, although during the course of the treatment, a major drop in endogenic testosterone unexpectedly occurred. In this case, compound 12 according to the invention induced in a dose-dependent way an increase of the weights of the seminal vesicles and prostates beyond the level of intact controls. A corresponding effect even in the case of extremely high dosage did not occur under MENT. Superior antigonadotropic effects of compound 12 according to the invention also could be ensured. Relative to the reduction of HDL, however, MENT is even more effective than the substances according to the invention. Compound 5 according to the invention is distinguished by especially small changes in hepatic parameters.

Description of the Test

Method: Adult male rats of above 300 g of body weight are treated orally over 6 weeks with MENT and compounds 12 or 5 according to the invention. At the end of the test, the animals are sacrificed, and the hormonal effects of the test substances are evaluated (weight of the testes and the accessory sex glands, reduction of the gonadotropic hormones and testosterone in the plasma during the course of the test, parameters of hepatic actions: effects on HDL lipoproteins).

The sulfamoyl benzoates of the androgens according to the invention are, as could be shown in Tests (I-III), clearly superior to the corresponding androgens with respect to oral androgenic and antigonadotropic activity. Effects that reflect the influence of the liver functions clearly outweigh the therapeutically desired androgenic effects.

Study of the Erythrocyte Bond

Compounds 5 and 12 according to the invention as well as compounds 1 and 11 according to the invention were studied, moreover, with respect to the binding to erythrocytes.

For the m-substituted compounds, significantly weaker bonds to the erythrocytes could be detected. The 4′-sulfamoyl benzoates, however, show a stronger bond.

In this case, however, it was unexpected that, e.g., the m-substituted compound 12 shows a higher activity in the Hershberger Test compared to the p-substituted compound 11 despite lower bond strength to the erythrocytes. The data in Table 1 are to illustrate the binding to erythrocytes of selected compounds according to formula (I).

TABLE 1
Binding of Selected Compounds to Erythrocytes
and Specific Erythrocyte/Plasma Ratios

| | | Distribution |
|---|---|---|
| | | Ratio with |
| | RBA in | Erythrocytes/ |
| Substance/Example | Erythrocytes | Plasma |
| Estradiol-3-sulfamate | 100 | 48 |
| (1) | 48 | 9 |
| (5) | 1.3 | 2 |
| (8) | 24 | 7 |
| (14) | 3.4 | 4 |
| (4) | 0.8 | 2 |
| (18) | 1.4 | 2 |
| (12) | 2.1 | 3 |

Principle of the Test and Test Description:

The SO₂—NH₂ group of the compounds according to the invention can result in a concentration in erythrocytes by binding to carboanhydrases. The displacement of estradiol-3-sulfamate from the erythrocyte bond by test substances is measured. Test preparation: human blood is mixed with a mixture that consists of ¹⁴C-labeled and unlabeled estradiol sulfamate. The erythrocytes are saturated at the selected working point. The distribution in plasma/erythrocytes is 40:60. A second blood sample is mixed with a mixture that consists of ¹⁴C-labeled estradiol sulfamate and unlabeled test substance. The relative binding affinity is calculated from the proportion of ¹⁴C-labeled estradiol sulfamate in plasma: higher proportion=strong displacement of ¹⁴C-estradiol sulfamate from the erythrocytes by the test substance=high binding affinity of the test substances to the erythrocytes.

Determination of the Erythrocyte/Plasma Distribution Ratio

Freshly obtained, heparinized blood is mixed with a defined amount of test substance. The concentration in the plasma obtained therefrom is measured. The erythrocyte/plasma distribution ratio is calculated from the measured concentration of the entire substance in plasma and the concentration that is used.

Surprisingly enough, in all cases a bond to the carboanhydrase (CA I) that is found in the erythrocytes nevertheless could be shown (Table 2). It is to be expected, therefore, that the substances according to the invention also have therapeutically relevant effects as carboanhydrase inhibitors. The bond to erythrocytes that is induced by the affinity to carboanhydrase is important for properties as androgen. This bond is essential for a reduced extraction of the orally administered substance in the first liver passage. High or low affinity to the erythrocytic carboanhydrase, faster or delayed release from this depot, and subsequent hydrolysis determine the therapeutic usability of the compounds according to the invention.

It was found, unexpectedly, that the compounds according to the invention reach therapeutically relevant levels with lower dosages if the ratio of the concentration of the erythrocytes/substance in the plasma-unlike what is to be expected according to DE 100 27 887.6 A1—is less than 10. By the compounds according to the invention, the possibility is opened up that higher shorter-term or uniformly low and longer-lasting hormone levels can be achieved with the same absolute amount of substance administered. As a result, active strengths and durations of action are varied and make possible a therapy that is matched to the individual organism.

TABLE 2
IC₅₀ Inhibiting Values of Human Carboanhydrase I

| | CAI |
|---|---|
| Inhibitor | IC₅₀ (nM) |
| (1) | 370 |
| (5) | 880 |
| (11) | 200-400 |
| (12) | 600 |
| Acetazolamide | 1200 |
| (known CA inhibitors) | 1900⁽¹²⁾ |

Principle of the Test and Test Description:

Carboanhydrases catalyze the CO₂ hydration.

Test preparation: A constant CO₂ stream is directed by a buffer that was mixed with carboanhydrase I. The time that is required to reduce the pH within defined limits is a measuring parameter. This parameter reflects the formation of H₂CO₃ in the medium. IC₅₀ inhibiting values are determined by test substances being pipetted into the test preparation. In the concentration areas that are examined, the test substances cause no inhibition to complete inhibition of the above-mentioned enzymes.

The test results of the in-vivo tests (Tests I-III) and the possibilities shown for variation of active strength and duration of action of a therapy matched to the individual organism open up many possible applications for birth control and hormone replacement therapy (HRT) in men and women as well as the treatment of hormonally induced diseases in men and women.

Subjects of this invention are therefore also pharmaceutical compositions that contain at least one compound of general formula (I) or a corresponding salt, optionally together with another steroidal active ingredient as well as with pharmaceutically compatible adjuvants and vehicles. Preferred active ingredients for this purpose are GnRH analogs, gestagens, antigestagens and glucocorticoids.

These pharmaceutical compositions and pharmaceutical agents can preferably be used for oral administration, but also for rectal, vaginal, subcutaneous, percutaneous, intravenous, buccal, transdermal or intramuscular administration. In addition to commonly used vehicles and/or diluents, they contain at least one compound of general formula (I) or salts thereof.

The pharmaceutical agents of the invention are produced with the commonly used solid or liquid vehicles or diluents and the commonly used pharmaceutical-technical adjuvants corresponding to the desired type of administration with a suitable dosage in a known way. The preferred preparations exist in a form for dispensing that is suitable for oral adminstration. Such forms for dispensing are, for example, tablets, film tablets, coated tablets, capsules, pills, powders, solutions or suspensions or else depot forms.

Of course, parenteral preparations such as injection solutions are also considered. In addition, for example, suppositories and agents for vaginal administration can also be mentioned as preparations.

Corresponding tablets can be obtained, for example, by mixing active ingredient with known adjuvants, for example inert diluents such as dextrose, sugar, sorbitol, mannitol, polyvinylpyrrolidone, explosives such as corn starch or alginic acid, binders such as starch or gelatins, lubricants such as manganese stearate or talc and/or agents for achieving a depot effect such as carboxylpolymethylene, carboxylmethyl cellulose, cellulose acetate phthalate or polyvinyl acetate. The tablets can also consist of several layers.

Coating cores, which are produced analogously to the tablets, with agents that are commonly used in tablet coatings, for example polyvinyl pyrrolidone or shellac, gum Arabic, talc, titanium oxide or sugar, can accordingly produce coated tablets. In this case, the shell of the coated tablet can also consist of several layers, whereby the adjuvants that are mentioned above in the tablets can be used.

Solutions or suspensions with the compounds of general formula (I) according to the invention can contain additional taste-improving agents such as saccharine, cyclamate or sugar, as well as, e.g., flavoring substances such as vanilla or orange extract. In addition, they can contain suspending adjuvants such as sodium carboxy methyl cellulose or preservatives, such as p-hydroxybenzoates.

The compounds of general formula (I) that contain capsules can be produced, for example, by the compound(s) of general formula (I) being mixed with an inert vehicle such as lactose or sorbitol and encapsulated in gelatin capsules.

Suitable suppositories can be produced, for example, by mixing with vehicles that are provided for this purpose, such as neutral fats or polyethylene glycol or derivatives thereof.

The compounds according to the invention are steroid prodrugs and have androgenic activity. The compounds according to the invention are especially suitable for oral administration. The therapeutically relevant steroid is released from the compound according to the invention or its salt by ester cleavage.

The compounds according to the invention are distinguished by a weak erythrocytic bond associated with a good androgenic activity. The factor that is used in this case as a yardstick for the binding or concentration in the erythrocytes is known from DE 100 27 887.6 A1 and is less than 10 in the compounds according to the invention.

The compounds according to the invention allow the therapeutic administration of the androgenically active compound in the form of an oral therapy and are not suitable for this type of therapy because of insufficient bioavailabity, e.g., that of testosterone, 5α-DHT or MENT.

The compounds according to the invention are suitable for substitution therapy, especially for substitution therapy in combination with antigonadotropic and antifertile strategies in males in connection with a GnRH-analog, a gestagen or other therapies that result in suppressing the endogenic androgen secretion.

The compounds according to the invention are also suitable for the substitution therapy in combination with glucocorticoids or other therapies that result in the suppression of the adrenal androgen secretion.

The compounds according to the invention are also suitable for the substitution therapy in women, especially after an age-typical drop in ovarian and adrenal androgen secretion.

Problems of liver compatibility are minimized by the compounds according to the invention. For conventional, orally administered androgens, especially for C-17 alkylated androgens, it is known that the liver is greatly stressed. The androgens that are contained in the compounds according to the invention do not stress the liver after their release.

The oral bioavailability of the androgen that is contained is increased by the compounds according to the invention. The entire hormone amount that has an effect on the organism is therefore reduced. The hormonal action on the liver in the first passage is reduced. With this, known disadvantageous effects of oral androgen therapy on the lipid metabolism (reduction of HDL cholesterol, increase of the atherogenic risk) and other hepatic secretion products are reduced.

Androgens differ in their hormonal action spectra and thus in their suitability for various therapeutic requirements. The compounds according to the invention allow the possibility of a differentiating androgen therapy by selection of the androgen that is contained.

The examples below are used for a more detailed explanation of the invention without being limited thereto.

General Synthesis Instructions

For the production of the androgens that are based on general formula (AII-CII), known steroid precursors can be used. The following steroid precursors can be used, for example:
testosterone, dihydrotestosterone, 19-nortestosterone, 7α-methyl-19-nortestosterone, 7α-methyl-11β-fluoro-1 g-nortestosterone and 3,3-dimethoxy-estr-5(10)-17-one (DD 79-213049), epiandrosterone, 5α-androst-2-en-17-one from epiandrosterone (U.S. Pat. No. 3,098,851), 7α-methyl-11β-methyl-19-nortestosterone, 7α-methyl-11β-methyltestosterone, oxandrolone, oral-turinabol, 17α-methyltestosterone, etc.

The functional groups that are contained in the starting materials for the steroid precursors can optionally be protected according to the methods that are known to one skilled in the art or can be converted into corresponding functional groups. Keto groups in the starting materials thus can be protected as ketals or thioketals according to methods that are known to one skilled in the art. 17-Keto compounds can be reduced to hydroxyl compounds according to methods that are known to one skilled in the art.

A) Coupling of the Steroidal Compound to Group Z

Variant 1

Reaction with Sulfamoylphenylcarboxylic Acids

An androgen is dissolved in a base, such as, e.g., pyridine. Corresponding amounts of a sulfamoylphenylcarboxylic acid are added to the solution, then an acid, such as, e.g., p-toluenesulfonic acid, and finally a carbodiimide, such as, e.g., dicyclohexylcarbodiimide, are added. The reaction mixture is stirred until the reaction is completed. Then, water is added, and it is acidified with an acid, such as, e.g., 10% HCl. The precipitate is filtered off, washed with water and NaHCO₃ solution and dried. The residue is extracted with an organic solvent, such as, e.g., ethyl acetate, the organic phase is washed and dried with a desiccant, such as, e.g., MgSO₄. After filtration, it is concentrated by evaporation and chromatographed on silica gel. Corresponding androgen sulfamoyl benzoates are obtained.

Variant 2

Reaction with Sulfamoylphenylcarboxylic Acid Chlorides

An androgen is dissolved in a base, such as, e.g., pyridine. The corresponding amount of a sulfamoylphenylcarboxylic acid chloride is added to the solution. The reaction mixture is stirred until the reaction is completed. Then, water is added, and it is acidified with an acid, such as, e.g., 10% HCl. It is extracted with an organic solvent, such as, e.g., ethyl acetate, the organic phase is washed, and it is dried with a desiccant, such as, e.g., MgSO₄. After filtration, it is concentrated by evaporation and chromatographed on silica gel. Corresponding androgen sulfamoyl benzoates are obtained.

Variant 3

Reaction with Chlorosulfonylphenylcarboxylic Acid Chlorides

An androgen is dissolved in a base, such as, e.g., pyridine, and an organic solvent, such as, e.g., chloroform, and cooled. The corresponding amount of a chlorosulfonylphenylcarboxylic acid chloride is added to the solution. The reaction mixture is stirred at room temperature until the reaction is completed. Then, the reaction mixture is stirred into concentrated ammonia solution. The mixture is concentrated by evaporation and acidified with an acid, such as, e.g., 10% HCl. The precipitate is suctioned off, washed with water, dried and chromatographed on silica gel. Corresponding androgen sulfamoyl benzoates are obtained.

Variant 4

Reaction with 2-Sulfophenylcarboxylic Acid-Cyclo-Anhydride

An androgen is dissolved in an organic solvent, such as, e.g., chloroform. After 2-sulfophenylcarboxylic acid-cyclo-anhydride is added, it is stirred at elevated temperatures under a cover gas. Then, it is cooled and mixed with a concentrated ammonia solution, such as, e.g., methanolic ammonia solution. The solvent is distilled off, and the residue is chromatographed on silica gel. 2′-Sulfophenylcarboxylic acid ester-ammonium salts of corresponding androgens, which are dissolved under a cover gas in an organic solvent, such as, e.g., CHCl₃, are obtained. A corresponding amount of a chlorinating agent, such as, e.g., PCl₅ or SOCl₂, is added in portions. The reaction mixture is stirred optionally at higher temperatures and then added briefly in concentrated NH₃ solution. The mixture is concentrated by evaporation, the precipitated substance is filtered off, washed with water, dried and chromatographed on silica gel. 2′-Sulfamoylphenylcarboxylic acid ester of corresponding androgens is obtained.

Variant 5

Reaction to Form Sulfamides (NH₂SO₂NH—)

The reaction to form the sulfamides according to the invention is carried out according to methods that are known to one skilled in the art for their production starting from corresponding amines by means of sulfamide, sulfamoyl chloride or aminosulfonyl isocyanate (P. O. Burke et al., J. Chem. Soc. Perk. Trans 2, 1984, 1851; M. Preiss et al. Chem. Ber., 1978, 1915: C.-H. Lee et al., J. Org. Chem., 1990, 6104).

For example, a corresponding amino benzoate in an organic solvent, such as, e.g., toluene, is reacted in the presence of a base, such as, e.g., NEt₃, with sulfamoyl chloride at temperatures of 20-60° C. The reaction mixture is stirred until the reaction is completed. Then, water is added, the precipitate is filtered off, washed with water and NaHCO₃ solution and dried. The substance is purified by chromatography on silica gel. Corresponding androgen sulfamoyl amino benzoates are obtained.

B) Synthesis of Group Z

### 2-Chloro-4-sulfamoylbenzoic Acid

Stage 1

10 g of 2-chloro-toluene-4-sulfonic acid-Na-salt x H₂O is added to 40 ml of thionyl chloride. After 5 ml of DMF is added, it is refluxed for 6 hours. The cold reaction mixture is added to 200 g of ice. The precipitated substance is washed with water and dried. 2-Chloro-toluene-4-sulfonic acid chloride is obtained.

¹H-NMR (DMSO-d₆): 2.32 (s, 3H, Me), 7.32-7.58 (m (superimposed), 3H, CH)

Stage 2

8 g of 2-chloro-toluene-4-sulfonic acid chloride is dissolved in 25 ml of CHCl₃ and slowly stirred into 100 ml of concentrated NH₃ solution. After 10 minutes of stirring at room temperature, the solution is concentrated by evaporation to one-half of its original volume. The substance is suctioned off, washed with water and dried. 2-Chloro-4-sulfamoyltoluene is obtained.

¹H-NMR (DMSO-d₆): 2.39 (s, 3H, Me), 7.44 (s, 2H, NH₂), 7.55-7.83 (m (superimposed), 3H, CH)

Stage 3

1.67 g of 2-chloro-4-sulfamoyltoluene is introduced into 70 ml of water. After 5 g of KMnO₄ and 0.5 ml of saturated NaHCO₃ solution are added, it is refluxed for 2 hours. After 2 ml of MeOH is added, the manganese dioxide that is produced is filtered off, and the solution is concentrated by evaporation to one-half of its original volume. After acidification with 10% HCl, the solution is cooled for 8 hours until crystallization is completed. Then, it is suctioned off, washed with water and dried. 2-Chloro-4-sulfamoylbenzoic acid is obtained.

¹H-NMR (DMSO-d₆): 7.66 (s, 2H, NH₂), 7.80-8.02 (m (superimposed), 3H, CH), 13.86 (s, 1H, COOH)

### 5-Sulfamoylisophthalic Acid

Stage 1

20 g of 5-sulfoisophthalic acid-Na-salt is boiled in 80 ml of thionyl chloride with the addition of 5 ml of DMF for 5 hours. The cold reaction mixture is added to 500 g of ice, and the precipitated substance is suctioned off, washed with water and dried. 5-Chlorosulfonyl-isophthalic acid dichloride is obtained.

¹H-NMR (CDCl₃): 8.98 (s, 2H, H-4,6), 9.11 (s, 1H, H-2)

Stage 2

10 g of 5-chlorosulfonylisophthalic acid dichloride is stirred into 150 ml of NH₃ solution in small portions. The solution is concentrated by evaporation, the precipitated substance is filtered off, washed with water and dried. 5-Sulfamoylisophthalic acid diamide is obtained.

¹H-NMR (DMSO-d₆): 7.51, 7.67, 8.22 (6H, NH₂), 8.43 (s, 2H, H-4,6), 8.53 (s, 1H, H-2)

Stage 3

1 g of 5-sulfamoylisophthalic acid diamide is suspended in 20 ml of 1,4-dioxane. 5 ml of 10% HCl is added, and the reaction mixture is heated for 3 hours to about 90° C. Then, the reaction mixture is evaporated to the dry state. The residue is chromatographed on silica gel. 5-Sulfamoylisophthalic acid-monoamide and 5-sulfamoylisophthalic acid are obtained.

### 4-Chlorosulfonylbenzoic Acid Chloride

15 g of 4-sulfonobenzoic acid-K-salt is dissolved in 100 ml of saturated ammonia solution. The solution is concentrated by evaporation, and the salt is dried on P₂O₅. 5 g of the salt is dissolved in 20 ml of SOCl₂. 0.3 ml of DMF is added to the reaction mixture and refluxed for 2 hours. It is allowed to cool off, toluene is added thereto for crystallization, and it is filtered off. The product is washed with toluene and dried. 4-Chlorosulfonylbenzoic acid chloride, which is used for further reactions, is obtained.

### SYNTHESIS EXAMPLES

### Example 1

### 3-Oxoandrost-4-en-17β-yl 4′-sulfamoyl benzoate (1)

Variant 1

1.0 g of testosterone is dissolved in 10 ml of pyridine. After 1.5 g of p-sulfamoylbenzoic acid, 200 mg of p-toluenesulfonic acid and 1.5 g of dicyclohexylcarbodiimide (DCC) are added, it is stirred for 48 hours at room temperature. Then, 20 ml of water is added. It is slightly acidified (pH=5) with 10% HCl. The precipitate is filtered off and washed 2× with saturated NaHCO₃ solution and water. The dried residue is extracted with ethyl acetate. The organic phase is washed with 10% NaHCO₃ solution and saturated NaCl solution, dried on MgSO₄, filtered, concentrated by evaporation and chromatographed on silica gel. 3-Oxoandrost-4-en-17β-yl 4′-sulfamoyl benzoate (1) is obtained.

Variant 2

1.0 g of testosterone is dissolved in 10 ml of pyridine. After 1.0 g of p-sulfamoylbenzoic acid chloride is added, it is stirred for 2 hours at room temperature. After 20 ml of water is added, it is slightly acidified (pH=5) with 10% HCl. Then, it is extracted with ethyl acetate. The organic phase is separated with 10% NaHCO₃ solution and washed with saturated NaCl solution, dried on MgSO₄, filtered, concentrated by evaporation and chromatographed on silica gel. 3-Oxoandrost-4-en-17β-yl 4′-sulfamoyl benzoate (1) is obtained.

¹H-NMR (DMSO-d₆): 0.95 (s, 3H, H-18), 1.17 (s, 3H, H-19), 4.80 (m, 1H, H-17α), 5.64 (s, 1H, H-4), 7.56 (s, 2H, NH₂), 7.92-8.15 (m, 4H, Ar).

### Example 2

### 3-Oxoandrostan-17β-yl 2′,4′-dichloro-5′-sulfamoyl benzoate (2)

The substance is obtained starting from 2,4-dichloro-5-sulfamoylbenzoic acid and testosterone analogously to Example 1 according to Variant 1.

¹H-NMR (DMSO-d₆): 0.90 (s, 3H, H-18), 1.16 (s, 3H, H-19), 4.82 (m, 1H, H-17α), 5.64 (s, 1H, H-4), 7.88 (s, 2H, NH₂), 8.02 (s, 1H, H—Ar), 8.37 (s, 1H, H—Ar).

### Example 3

### 3-Oxoandrost-4-en-17β-yl 2′-chloro-5′-sulfamoyl benzoate (3)

The substance is obtained starting from 2-chloro-5-sulfamoylbenzoic acid and testosterone analogously to Example 1 according to variant 1.

¹H-NMR (DMSO-d₆): 0.90 (s, 3H, H-18), 1.16 (s, 3H, H-19), 4.77 (m, 1H, H-17α), 5.64 (s, 1H, H-4), 7.56 (s, 2H, NH₂), 7.75-8.80 (m, 3H, H—Ar).

### Example 4

### 3-Oxoandrost-4-en-17β-yl 2′-methoxy-5′-sulfamoyl benzoate (4)

The substance is obtained starting from 2-methoxy-5-sulfamoylbenzoic acid and testosterone analogously to Example 1 according to Variant 1.

¹H-NMR (DMSO-d₆): 0.90 (s, 3H, H-18), 1.16 (s, 3H, H-19), 4.74 (m, 1H, H-17α), 5.64 (s, 1H, H-4), 7.33 (m, 1H, H—Ar), 7.35 (s, 2H, NH₂), 7.92-8.07 (m, 2H, H—Ar).

### Example 5

### 3-Oxoandrost-4-en-17β-yl 3′-sulfamoyl benzoate (5)

1.0 g of testosterone is dissolved in 2 ml of pyridine and 2 ml of CHCl₃. 1.0 ml of 3-chlorosulfonylbenzoic acid chloride is added to the reaction mixture at −20° C. while being stirred. Then, it is heated to room temperature and stirred for 15 minutes. The reaction solution is added to 25 ml of concentrated NH₃ solution and stirred for 15 minutes. Then, the organic mobile solvent is distilled off. It is slightly acidified (pH=5) with 10% hydrochloric acid. The precipitated substance is suctioned off, washed with 10% NaHCO₃ solution and water, dried and chromatographed on silica gel. 3-Oxoandrost-4-en-17β-yl 3′-sulfamoyl benzoate (5) is obtained.

¹H-NMR (CDCl₃): 0.98 (s, 3H, H-18), 1.21 (s, 3H, H-19), 4.88 (m, 1H, H-17α), 5.28 (s, 2H, NH₂), 5.74 (s, 1H, H-4), 7.33 (m, 1H, H—Ar), 7.60-8.60 (m, 4H, H—Ar).

### Example 6

### 3-Oxoandrost-4-en-17β-yl 2′-sulfamoyl benzoate (6)

Stage 1

3-Oxoandrost-4-en-17β-yl 2′-sulfobenzoate-Ammonium Salt

2.5 g of testosterone is dissolved in 10 ml of chloroform. After 3.5 g of 2-sulfobenzoic acid-cyclo-anhydride is added, it is stirred under a cover gas for 12 hours at 50° C. Then, it is cooled to 0° C. and mixed with a concentrated methanolic ammonia solution. The solvents are distilled off, and the residue is chromatographed on silica gel. 3-Oxoandrost-4-en-17β-yl 2′-sulfobenzoate ammonium salt is obtained.

¹H-NMR (DMSO-d₆): 0.81 (s, 3H, H-18), 1.15 (s, 3H, H-19), 4.67 (m, 1H, H-17α), 5.63 (s, 1H, H-4), 7.33 (m, 1H, H—Ar), 7.18-7.75 (m, 4H, H—Ar).

Stage 2

### 3-Oxoandrost-4-en-17β-yl 2′-sulfamoyl benzoate (6)

3.95 g of 3-oxoandrost-4-en-17β-yl 2′-sulfobenzoate ammonium salt is dissolved in 160 ml of CHCl₃ under a cover gas. 6.0 g of PCl₅ is added in portions within 1 hour. The reaction mixture is stirred for 12 hours at room temperature and then added to 600 ml of concentrated NH₃ solution. The mixture is concentrated by evaporation, the precipitated substance is filtered off, washed with water, dried and chromatographed on silica gel. 3-Oxoandrost-4-en-17β-yl 2′-sulfamoyl benzoate (6) is obtained.

¹H-NMR (CDCl₃): 0.94 (s, 3H, H-18), 1.20 (s, 3H, H-19), 4.90 (m, 1H, H-17α), 5.73 (s, 1H, H-4), 5.80 (s, 2H, NH₂), 7.58-8.15 (m, 4H, H—Ar).

### Example 7

### 3-Oxoestr-4-en-17β-yl 3′-sulfamoyl benzoate (7)

The substance is obtained starting from nandrolone, 3-chlorosulfonylbenzoic acid chloride and ammonia analogously to Example 5.

¹H-NMR (DMSO-d₆): 0.98 (s, 3H, H-19), 4.82 (m, 1H, H-17α), 5.74 (s, 1H, H-4), 7.54 (s, 2H, NH₂), 7.68-8.38 (m, 4H, H—Ar).

### Example 8

### 3-Oxoestr-4-en-17β-yl 4′-sulfamoyl benzoate (8)

The substance is obtained starting from nandrolone and p-sulfamoylbenzoic acid analogously to Example 1, Variant 1.

¹H-NMR (DMSO-d₆): 0.97 (s, 3H, H-19), 4.80 (m, 1H, H-17α), 5.74 (s, 1H, H-4), 7.57 (s, 2H, NH₂), 7.92-8.15 (m, 4H, H—Ar).

### Example 9

### 3-Oxoandrostan-17β-yl 3′-sulfamoyl benzoate (9)

The substance is obtained starting from 17β-hydroxy-5α-androstan-3-one, 3-chlorosulfonylbenzoic acid chloride and ammonia analogously to Example 5.

¹H-NMR (DMSO-d₆): 0.92 (s, 3H, H-18), 1.00 (s, 3H, H-19), 4.79 (m, 1H, H-17α), 7.54 (s, 2H, NH₂), 7.68-8.38 (m, 4H, H—Ar).

### Example 10

### 3-Oxoandrostan-17β-yl 4′-sulfamoyl benzoate (10)

The substance is obtained starting from 17β-hydroxy-5α-androstan-3-one and p-sulfamoylbenzoic acid analogously to Example 1, Variant 1.

¹H-NMR (DMSO-d₆): 0.92 (s, 3H, H-18), 0.99 (s, 3H, H-19), 4.78 (m, 1H, H-17α), 7.56 (s, 2H, NH₂), 7.90-8.13 (m, 4H, H—Ar).

### Example 11

### 3-Oxo-7α-methylestr-4-en-17β-yl 4′-sulfamoyl benzoate (11)

The substance is obtained starting from 17β-hydroxy-7α-methylestr-4-en-3-one (MENT) and p-sulfamoylbenzoic acid analogously to Example 1, Variant 1.

¹H-NMR (DMSO-d₆): 0.75 (d, 3H, 7α-Me), 0.98 (s, 3H, H-19), 4.82 (m, 1H, H-17α), 5.72 (s, 1H, H-4), 7.55 (s, 2H, NH₂), 7.92-8.15 (m, 4H, H—Ar).

### Example 12

### 3-Oxo-7α-methylestr-4-en-17β-yl 3′-sulfamoyl benzoate (12)

The substance is obtained starting from 17β-hydroxy-7α-methylestr-4-en-3-one (MENT), chlorosulfonylbenzoic acid chloride and ammonia analogously to Example 5.

¹H-NMR (DMSO-d₆): 0.75 (d, 3H, 7α-Me), 0.98 (s, 3H, H-19), 4.83 (m, 1H, H-17α), 5.74 (s, 1H, H-4), 7.57 (s, 2H, NH₂), 7.68-8.38 (m, 4H, H—Ar).

### Example 13

### 3-Oxo-7α-methylestr-4-en-17β-yl 2′-chloro-5′-sulfamoyl benzoate (13)

The substance is obtained starting from 17β-hydroxy-7α-methylestr-4-en-3-one (MENT) and 2-chloro-5-sulfamoylbenzoic acid analogously to Example 1, Variant 1.

¹H-NMR (DMSO-d₆): 0.74 (d, 3H, 7α-CH₃), 0.93 (s, 3H, H-19), 4.84 (m, 1H, H-17α), 5.72 (s, 1H, H-4), 7.60 (s, 2H, NH₂), 7.75-8.18 (m, 4H, H—Ar).

### Example 14

### 3-Oxoandrost-4-en-17β-yl 4′-sulfamoylphenylpropionate (14)

The substance is obtained starting from 3-(p-sulfamoylphenyl)propionic acid and testosterone analogously to Example 1 according to Variant 1.

¹H-NMR (DMSO-d₆): 0.74 (s, 3H, CH₃), 1.14 (s, 3H, CH₃), 2.66 (t, 2H, CH₂), 2.92 (t, 2H, CH₂), 4.50 (m, 1H, H-17α), 5.62 (s, 1H, H-4), 7.28 (s, 2H, NH₂), 7.37-7.72 (m, 4H, H—Ar).

### Example 15

### 3-Oxo-5α-androst-1-en-17β-yl 3′-sulfamoyl benzoate (15)

The substance is obtained starting from 17β-hydroxy-5α-androst-1-en-3-one, 3-chlorosulfonylbenzoic acid chloride and ammonia analogously to Example 5.

¹H-NMR (DMSO-d₆): 0.94 (s, 3H, H-18), 0.99 (s, 3H, H-19), 4.82 (m, 1H, H-17α), 5.74 (s, 1H, H-2), 7.21 (s, 1H, H-1), 7.54 (s, 2H, NH₂), 7.70-8.38 (m, 4H, H—Ar).

### Example 16

### 3-Oxo-4-chloro-17α-methylandrosta-1,4-dien-17β-yl 3′-Sulfamoyl Benzoate (16)

The substance is obtained starting from 4-chloro-17α-methyl-17β-hydroxyandrosta-1,4-dien-3-one, 3-chlorosulfonylbenzoic acid chloride and ammonia analogously to Example 5.

¹H-NMR (DMSO-d₆): 1.02 (s, 3H, H-18), 1.31 (s, 3H, H-19), 1.48 (s, 3H, CH₃-17α), 6.30 (d, 1H, H-2), 7.31 (d, 1H, H-1), 7.53 (s, 2H, NH₂), 7.70-8.38 (m, 4H, H—Ar).

### Example 17

### 3-Oxo-7α-methyl-11β-fluoro-estr-4-en-17β-yl 3′-sulfamoyl Benzoate (18)

The substance is obtained starting from 17β-hydroxy-7α-methyl-11β-fluoro-estr-4-en-3-one, 3-chlorosulfonylbenzoic acid chloride and ammonia according to Variant 3 analogously to Example 5.

¹H-NMR (DMSO-d₆): 0.77 (d, 3H, 7α-Me), 1.07 (s, 3H, H-18), 4.85 (m, 1H, H-17α), 5.78 (s, 1H, H-4), 7.55 (s, 2H, NH₂), 7.70-8.38 (m, 4H, H—Ar).

### Example 18

### 3-Oxoandrost-4-en-17β-yl 2′-hydroxy-5′-sulfamoyl Benzoate (18)

The substance is obtained starting from 2-hydroxy-5-sulfamoylbenzoic acid and testosterone analogously to Example 1 according to Variant 1.

¹H-NMR (DMSO-d₆): 0.94 (s, 3H, H-18), 1.17 (s, 3H, H-19), 4.81 (m, 1H, H-17α), 5.64 (s, 1H, H-4), 7.14 (m, 1H, H—Ar), 7.36 (s, 2H, NH₂), 7.90-8.2 (m, 2H, H—Ar), 11.03 (s, 1H, OH).

### Example 19

### 3-Oxo-4-chloroandrost-4-en-17β-yl 3′ sulfamoyl Benzoate (19)

The substance is obtained starting from 3-oxo-4-chloro-17β-hydroxyandrost-4-ene and 3-chlorosulfonylbenzoic acid chloride analogously to Example 5 according to Variant 3.

¹H-NMR (DMSO-d₆): 0.95 (s, 3H, H-18), 1.23 (s, 3H, H-19), 4.81 (m, 1H, H-17α), 7.55 (s, 2H, NH₂), 7.75 (m, 1H, H—Ar), 8.05-8.28 (m, 2H, H—Ar), 8.38 (s, 1H, H—Ar)

### Example 20

### 3-Oxo-4-chloroandrosta-1,4-dien-17β-yl 3′-sulfamoyl Benzoate (20)

The substance is obtained starting from 3-oxo-4-chloro-17β-hydroxyandrosta-1,4-diene and 3-chlorosulfonylbenzoic acid chloride analogously to Example 5 according to Variant 3.

¹H-NMR (DMSO-d₆): 4.81 (m, 1H, H-17α), 7.55 (s, 2H, NH₂), 7.75 (m, 1H, H-Ar), 8.05-8.28 (m, 2H, H—Ar), 8.38 (s, 1H, H—Ar).

### Example 21

### 3-Oxo-17β-hydroxyestr-4-en-yl 3′-sulfamoyl benzoate (21)

Stage 1: 4-Thexyldimethylsilyloxy-17β-acetoxyestr-4-en-3-one

5.9 g of 4-hydroxy-17β-acetoxyestr-4-en-3-one is reacted in 58 ml of DMF with 12 g of imidazole and 15 ml of thexyldimethylsilyl chloride for 2 hours at 35° C. 500 ml of water is added to the reaction solution. Then, it is extracted with ethyl acetate, dried with MgSO₄, and concentrated by evaporation. The product is purified by chromatography on silica gel. 4-Thexyldimethylsilyloxy-17β-acetoxyestr-4-en-3-one is obtained.

¹H-NMR (CDCl₃): 0.14 (s, 6H, SiMe), 0.65-0.88 (m (upper), 15H, thexyl, H-18), 2.04 (s, 3H, OAc), 4.60 (m, 1H, H-17α).

Stage 2: 4-Thexyldimethylsilyloxy-17β-hydroxyestr-4-en-3-one 2 g of 4-thexyldimethylsilyloxy-17β-acetoxyestr-4-en-3-one is dissolved in 50 ml of MeOH. After 2 g of K₂CO₃ and 2 ml of water are added, it is stirred for 2 hours at 30° C. 100 ml of water is added to the reaction solution, and the MeOH is distilled off to a very large extent. Then, it is extracted with ethyl acetate, dried with MgSO₄ and concentrated by evaporation. The product is purified by chromatography on silica gel. 4-Thexyldimethylsilyloxy-17β-hydroxyestr-4-en-3-one is obtained.

Stage 3: 4-Thexyldimethylsilyloxy-17β-[perhydropyran-2-yl)oxy]estr-4-en-3-one

1 g of 4-thexyldimethylsilyloxy-17β-hydroxyestr-4-en-3-one is reacted in 16 ml of CH₂Cl₂ with 1.8 ml of dihydropyran and 80 mg of pyridinium tosylate for 2 hours. 10 ml of saturated Na₂CO₃ solution is added to the reaction solution. Then, it is extracted with CH₂Cl₂, dried with MgSO₄ and concentrated by evaporation. The product is purified by chromatography on silica gel. 4-Thexyldimethylsilyloxy-17β-[perhydropyran-2-yl)oxy]estr-4-en-3-one is obtained.

Stage 4: 4-Hydroxy-17β-[perhydropyran-2-yl)oxy]estr-4-en-3-one

1 g of 4-thexyldimethylsilyloxy-17β-[perhydropyran-2-yl)oxy]estr-4-en-3-one is reacted in 20 ml of THF with 300 mg of TBAF for 1 hour at room temperature. 10 ml of water is added to the reaction solution. Then, it is extracted with ethyl acetate, dried with MgSO₄ and concentrated by evaporation. The product is purified by chromatography on silica gel. 4-Hydroxy-17β-[perhydropyran-2-yl)oxy]ester-4-en-3-one is obtained.

Stage 5: 3-Oxo-17β-[perhydropyran-2-yl)oxy]estr-4-en-4-yl 3′-sulfamoyl Benzoate (22)

The substance is obtained starting from 4-hydroxy-17β-[perhydropyran-2-yl)oxy]estr-4-en-3-one and 3 chlorosulfonyl benzoic acid chloride analogously to Example 5 according to Variant 3.

Stage 6: 3-Oxo-17β-hydroxyestr-4-en-4-yl 3′-sulfamoyl Benzoate (21)

500 mg of 3-oxo-17β-[perhydropyran-2-yl)oxy]estr-4-en-4-yl 3′-sulfamoyl benzoate is dissolved in 25 ml of acetone and reacted with 3 ml of 10% HCl for 1 hour at room temperature. 10 ml of saturated Na₂CO₃ solution is added to the reaction solution. Then, acetone is distilled off and extracted with ethyl acetate, dried with MgSO₄ and concentrated by evaporation. The product is purified by chromatography on silica gel. 3-Oxo-17β-hydroxyestr-4-en-4-yl 3′-sulfamoyl benzoate (21) is obtained.

¹H-NMR (DMSO-D₆): 0.71 (s. 3H, H-18), 3.46 (m, 1H, H-17α), 4.50 (d, 1H, 4-OH), 7.57 (s, 2H, NH₂), 7.75-8.44 (m, 4H, H—Ar).

### Example 22

### 3-Oxoandrost-4-en-17β-yl 2′-chloro-4′-sulfamoylbenzoate (23)

1.0 g of testosterone is dissolved in 20 ml of pyridine. After 1.5 g of 2-chloro-4-sulfamoylbenzoic acid, 250 mg of p-toluenesulfonic acid and 1.5 g of DCC are added, it is stirred for 48 hours at room temperature. Then, 100 ml of water and 30 ml of CHCl₃ are added. It is slightly acidified (pH=5) with 10% HCl. The precipitate is filtered off and rewashed with CHCl₃. The organic phase is separated with 10% NaHCO₃ solution and washed with saturated NaCl solution, dried on MgSO₄, filtered, concentrated by evaporation and chromatographed on silica gel. 3-Oxoandrost-4-en-17β-yl 2′-chloro-4′-sulfamoylbenzoate is obtained.

¹H-NMR (DMSO-d₆): 0.89 (s, 3H, H-18), 1.16 (s, 3H, H-19), 4.80 (t, 1H, H-17), 5.64 (s, 1H, HC═), 7.64 (s, 2H, NH₂)

### Example 23

### 3-Oxoandrost-4-en-17β-yl 3′-carboxy-5′-sulfamoylbenzoate (24)

1.0 g of testosterone is dissolved in 3.5 ml of pyridine. After 1.1 g of 5-sulfamoylisophthalic acid and 880 mg of EDC are added, it is stirred for 48 hours at room temperature. Then, 10 ml of water is added. It is acidified with 10% HCl. The precipitate is filtered off, washed with water and dried. It is chromatographed on silica gel. 3-Oxoandrost-4-en-17β-yl 3′-carboxy-4′-sulfamoylbenzoate is obtained.

¹H-NMR (DMSO-d₆): 0.96 (s, 3H, H-18), 1.17 (s, 3H, H-19), 4.84 (t, 1H, H-17), 5.64 (s, 1H, HC═), 7.62 (s, 2H, NH₂), 7.72, 8.45 (2H, NH₂)

### Example 24

### 3-Oxoandrost-4-en-17β-yl 3′-carbamido-5′-sulfamoylbenzoate (25)

1.0 g of testosterone is dissolved in 3.5 ml of pyridine. After 1.1 g of 5-sulfamoylisophthalic acid monoamide and 880 mg of EDC are added, it is stirred for 48 hours at room temperature. Then, 10 ml of water is added. It is acidified with 10% HCl. The precipitate is filtered off, washed with water, and dried. It is chromatographed on silica gel. 3-Oxoandrost-4-en-17β-yl 3′-carbamido-4′-sulfamoylbenzoate is obtained.

¹H-NMR (DMSO-d₆): 0.95 (s, 3H, H-18), 1.17 (s, 3H, H-19), 4.83 (t, 1H, H-17), 5.64 (s, 1H, HC═), 7.55 (s, 2H, NH₂), 8.35-8.64 (3s, 3H, CH)

Literature

(1) Moordian, A. D., Morley, J. E., and Korenman, S. G. (1987) Biological Actions of Androgens. Endocr. Rev. 8, 1-27.
(2) Rommerts, F. F. G. (1990) Testosterone: An Overview of Biosynthesis, Transport, Metabolism and Nongenomic Actions. In: Nieschlag, E., and Behre, H. M. (eds.) Testosterone: Action, Deficiency, Substitution. 2^{nd} Edition (1998), Springer, Berlin Heidelberg, Chapter 1, 1-31; Wilson, J. D., Griffin, J. E., Russell, D. W. (1993) Steroid 5α-Reductase 2 Deficiency. Endocr. Rev. 14, 577-93.
(3) Jost, A. (1946) Recherches sur la differenciation sexuelle de l″embryon de lapin: III. Role des gonades foetales dans la differenciation sexuelle somatique. [Research on the Sexual Differentiation of the Rabbit Embryo: III. Role of Fetal Gonads in Somatic Sexual Differentiation] Arch Anat micr Morph exp; 36, 271-315; Neumann, F., Berswordt-Wallrabe, R., Elger, W., Steinbeck, H., Hahn, J. D., et al. (1970) Aspects of Androgen-Dependent Events as Studied by Antiandrogens. Recent Prog. Horm. Res. 26, 337-410; Neumann, F., Elger, W., Kramer, M. (1966) Development of a Vagina in Male Rats by Inhibiting Androgen Receptors with an Anti-Androgen During the Critical Phase of Organogenesis. Endocrinology 78(3), 628-32.
(4) Liu, P. Y., and Handelsman, D. J. (1990) Androgen Therapy in Non-gonadal Disease. In: Nieschlag, E., and Behre, H. M. (eds.) Testosterone: Action, Deficiency, Substitution. 2^{nd} Edition (1998), Springer, Berlin Heidelberg, Chapter 17, 473-512.
(5) Quigley, C. A. (1990) The Androgen Receptor: Physiology and Pathophysiology. In: Nieschlag, E., and Behre, H. M. (eds) Testosterone: Action, Deficiency, Substitution. 2^{nd} Edition (1998), Springer, Berlin Heidelberg, Chapter 2, 33-106.
(6) Cardim, H. J. P., Lopes, C. M. C., Giannella-Neto, D., da Fonseca, A. M., and Pinotti, J. A. (2001), The Insulin-like Growth Factor-I System and Hormone Replacement Therapy. Fertility and Sterility 75(2), 282-7; Liu, P. Y., and Handelsman, D. J. (1990) Androgen Therapy in Non-Gonadal Disease. In: Nieschlag, E., and Behre, H. M. (eds.) Testosterone: Action, Deficiency, Substitution. 2^{nd} Edition (1998), Springer, Berlin Heidelberg, Chapter 17, 473-512.
(7) Rommerts, F. F. G. (1990) Testosterone: An Overview of Biosynthesis, Transport, Metabolism and Nongenomic Actions. In: Nieschlag, E., and Behre, H. M. (eds.) Testosterone: Action, Deficiency, Substitution. 2^{nd} Edition (1998), Springer, Berlin Heidelberg, Chapter 1, 1-31.
(8) Moordian, A. D., Morley, J. E., and Korenman, S. G. (1987) Biological Actions of Androgens. Endocr. Rev. 8, 1-27.
(9) Arlt, W., Callies, F., and Allolio, B. (2000), DHEA Replacement in Women with Adrenal Insufficiency—Pharmacokinetics, Bioconversion and Clinical Effects on Well-Being, Sexuality and Cognition. Endocr. Res. 26(4), 505-11.
(10) Liu, P. Y., and Handelsman, D. J. (1990) Androgen Therapy in Non-Gonadal Disease. In: Nieschlag, E., and Behre, H. M. (eds) Testosterone:

Action, Deficiency, Substitution. 2^{nd} Edition (1998), Springer, Berlin Heidelberg, Chapter 17, 473-512.
(11) Nieschlag, E., and Behre, H. M. (1990) Pharmacology and Clinical Uses of Testosterone. In: Nieschlag, E., and Behre, H. M. (eds) Testosterone: Action, Deficiency, Substitution. 2^{nd} Edition (1998), Springer, Berlin Heidelberg, Chapter 10, 293-328.
(12) C. Ladolfi, M. Marchetti, G. Ciocci, and C. Milanese, Journal of Pharmacological and Toxicological Methods 38, 169-172 (1997).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosure of all applications, patents and publications, cited herein and of corresponding U.S. Provisional Application Ser. No. 60/572,873, filed May 21, 2004, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

[image]

[image]

1. Steroid prodrugs of general formula (1) 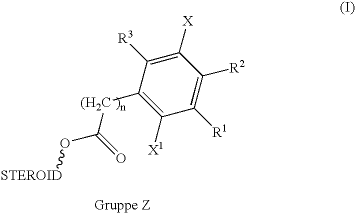
in which n is a number 0-4,
R¹ is a radical —SO₂NH₂ or —NHSO₂NH₂,
whereby R², R³ and X, X¹ stand for a hydrogen atom, a halogen atom, a nitrile group, a nitro group, a C₁₋₅-alkyl group, a CₚF₂ₚ₊₁ group with p=1-3, a group OC(O)R²⁰, COOR²⁰, OR²⁰, C(O)NHR²⁰, or OC(O)NH—R²¹, whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and
R²⁰ in addition can mean a hydrogen, or
R² is a radical —SO₂NH₂ or —NHSO₂NH₂,
whereby R², R³ and X, X¹ stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and R²⁰ in addition can mean a hydrogen, or
R³ is a radical —SO₂NH₂ or —NHSO₂NH₂,
whereby R², R³ and X, X¹ stand for a hydrogen atom, a halogen atom, a nitrile group, a nitro group, a C₁₋₅-alkyl group, a
CₚF₂ₚ₊₁ group with p=1-3, a group OC(O)—R²⁰, COOR²⁰, OR²⁰, C(O)NHR²⁰, or OC(O)NH—R²¹,
whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and R²⁰ in addition can mean a hydrogen, and
STEROID stands for a steroidal ring system according to general formulas (AII-CII): 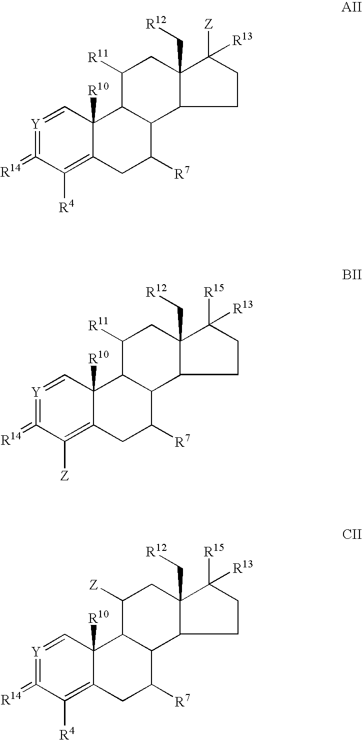
whereby
Y represents an oxygen atom or a carbon atom,
R⁴ represents a hydrogen atom, a halogen atom, a methyl, trifluoromethyl, hydroxy, tri(C₁₋₆-alkyl)silyloxy, C₁₋₅-alkoxy or a C₂₋₅-heterocycloalkyloxy group,
R⁷ represents a hydrogen atom, a methyl or ethyl group,
R¹⁰ represents a hydrogen atom, a methyl or ethyl group,
R¹¹ represents a halogen atom, a hydrogen atom, a hydroxy group, a methoxy group, a group OC(O)R²⁰, a methyl or ethyl group,
R¹² represents a hydrogen atom, a methyl group or an ethyl group,
R¹³ represents a hydrogen atom, a methyl, ethyl, ethinyl, trifluoromethyl, or pentafluoroethyl group,
R¹⁴ represents a hydrogen atom, an OH group, or an oxygen atom that is bonded via a double bond,
R¹⁵ represents a hydroxy, tri(C₁₋₆-alkyl)silyloxy, C₁₋₅-alkoxy group, a group OC(O)—R²⁰ or a C₂₋₅-heterocycloalkyloxy group,
whereby an additional double bond can be found in 4,5-position and if Y stands for a carbon atom, additional double bonds can be found in 1,2-position or if radical R¹⁴ is a hydrogen atom or an OH group in 2,3-position,
and their pharmaceutically acceptable salts.

2. Compounds according to claim 1, characterized in that n is 0, 1 or 2.

3. Compounds according to claim 1,
wherein R¹ represents a radical —SO₂NH₂ or —NHSO₂NH₂.

4. Compounds according to claim 3, wherein R' represents a group —SO₂NH₂.

5. Compounds according to claim 1, wherein either R¹, R² or R³ represents a group —SO₂NH₂.

6. Compounds according to claim 1, wherein
R¹, R², R³, if the latter do not represent —SO₂NH₂ or —NHSO₂NH₂, as well as X and X¹, independently of one another, stand for a hydrogen, fluorine, or chlorine atom or a hydroxy or a methoxy group.

7. Compounds according to claim 1, wherein STEROID stands for a steroidal ring system for general partial formulas AII and BII.

8. Compounds according to claim 1, wherein, independently of one another,
Y represents a carbon atom and/or
R⁴ represents a hydrogen atom, a chlorine atom, or a hydroxy group and/or
R⁷ represents a hydrogen atom or a methyl group and/or
R¹⁰ represents a hydrogen atom or a methyl group and/or
R¹¹ represents a hydrogen atom, a fluorine atom or a methyl group and/or
R¹² represents a hydrogen atom and/or
R¹³ represents a hydrogen atom or a methyl group,
R¹⁴ represents an oxygen atom and/or
R¹⁵ represents a hydroxy group or a group OC(O)R²⁰.

9. Compounds according to claim 1, namely
1) 3-Oxo-7α-methylestr-4-en-17β-yl 3′-sulfamoyl benzoate (12),
2) 3-Oxo-7α-methylestr-4-en-17β-yl 4′-sulfamoyl benzoate (11),
3) 3-Oxo-7α-methylestr-4-en-17β-yl 2′-chloro-5′-sulfamoyl benzoate (13),
4) 3-Oxo-7α-methylestr-4-en-17β-yl 2′,4′-dichloro-5′-sulfamoyl benzoate,
5) 3-Oxo-7α-methylestr-4-en-17β-yl 2′-methoxy-5′-sulfamoyl benzoate,
6) 3-Oxo-7α-methylestr-4-en-17β-yl 2′,3′-dimethoxy-5′-sulfamoyl benzoate,
7) 3-Oxoestr-4-en-17β-yl 3′-sulfamoyl benzoate (7),
8) 3-Oxoestr-4-en-17β-yl 4′-sulfamoyl benzoate (8),
9) 3-Oxoestr-4-en-17β-yl 2′,4′-dichloro-5′-sulfamoyl benzoate,
10) 3-Oxoestr-4-en-17β-yl 2′-chloro-5′-sulfamoyl benzoate,
11) 3-Oxoestr-4-en-17β-yl 2′-methoxy-5′-sulfamoyl benzoate,
12) 3-Oxoestr-4-en-17β-yl 2′,3′-dimethoxy-5′-sulfamoyl benzoate,
13) 3-Oxo-7α-methylandrost-4-en-17β-yl 3′-sulfamoyl benzoate,
14) 3-Oxo-7α-methylandrost-4-en-17β-yl 4′-sulfamoyl benzoate,
15) 3-Oxo-7α-methylandrost-4-en-17β-yl 2′-chloro-5′-sulfamoyl benzoate,
16) 3-Oxo-7α-methylandrost-4-en-17β-yl 2′,4′-dichloro-5′-sulfamoyl benzoate,
17) 3-Oxo-7α-methylandrost-4-en-17β-yl 2′-methoxy-5′-sulfamoyl benzoate,
18) 3-Oxo-7α-methylandrost-4-en-17β-yl 2′,3′-dimethoxy-5′-sulfamoyl benzoate,
19) 3-Oxoandrost-4-en-17β-yl 3′-sulfamoyl benzoate (1),
20) 3-Oxoandrost-4-en-17β-yl 4′-sulfamoyl benzoate (1),
21) 3-Oxoandrost-4-en-17β-yl 2′,4′-dichloro-5′-sulfamoyl benzoate,
22) 3-Oxoandrost-4-en-17β-yl 2′-chloro-5′-sulfamoyl benzoate,
23) 3-Oxoandrost-4-en-17β-yl 2′-methoxy-5′-sulfamoyl benzoate,
24) 3-Oxoandrost-4-en-17β-yl 2′,3′-dimethoxy-5′-sulfamoyl benzoate,
25) 3-Oxoandrostan-17β-yl 3′-sulfamoyl benzoate (9),
26) 3-Oxoandrostan-17β-yl 4′-sulfamoyl benzoate (10),
27) 3-Oxoandrost-4-en-17β-yl 2′,4′-dichloro-5′-sulfamoyl benzoate (2),
28) 3-Oxoandrost-4-en-17β-yl 2′-chloro-5′-sulfamoyl benzoate (3),
29) 3-Oxoandrost-4-en-17β-yl 2′-methoxy-5′-sulfamoyl benzoate (4)
30) 3-Oxoandrost-4-en-17β-yl 2′,3′-dimethoxy-5′-sulfamoyl benzoate,
31) 3-Oxo-4-chloro-17α-methylandrosta-1,4-dien-17-yl 3′-sulfamoyl benzoate (6),
32) 3-Oxo-4-chloro-17α-methylandrosta-1,4-dien-17β-yl 4′-sulfamoyl benzoate,
33) Androst-2-en-17β-yl 3′-sulfamoyl benzoate,
34) 3-Oxoandrost-4-en-17β-yl 2′-sulfamoyl benzoate (6),
35) Androst-2-en-17β-yl-4′-sulfamoyl benzoate,
36) 3-Oxo-7α-methyl-11β-fluoroestr-4-en-17β-yl 3′-sulfamoyl benzoate (17),
37) 3-Oxoandrost-4-en-17β-yl 4′-sulfamoylphenylpropionate (14),
38) 3-Oxo-5-androst-1-en-17β-yl 3′-sulfamoyl benzoate (15),
39) 3-Oxoandrost-4-en-17β-yl 2′-hydroxy-5′-sulfamoyl benzoate (18),
40) 3-Oxoandrostan-17β-yl 2′hydroxy-5′-sulfamoyl benzoate,
41) 3-Oxo-7α-methyl-11β-fluoroestr-4-en-17β-yl 2′-hydroxy-5′sulfamoyl benzoate,
42) 3-Oxoestr-4-en-17β-yl 2′-hydroxy-5′-sulfamoyl benzoate,
43) 3-Oxo-7α-methylestr-4-en-17β-yl 2′-hydroxy-5′-sulfamoyl benzoate,
44) 3-Oxo-4-chloroandrost-4-en-17β-yl 3′-sulfamoyl benzoate (19),
45) 3-Oxo-4-chloroandrosta-1,4-dien-17β-yl 3′-sulfamoyl benzoate (20),
46) 3-Oxo-4-hydroxyestr-4-en-17β-yl 3′-sulfamoyl benzoate,
47) 3-Oxo-4-hydroxyandrost-4-en-17β-yl 3′-sulfamoyl benzoate
48) 3-Oxo-17β-[(perhydropyran-2-yl)oxy]estr-4-en-4-yl 3′-sulfamoyl benzoate (22),
49) 3-Oxo-17β-hydroxyestr-4-en-4-yl 3′-sulfamoyl benzoate (21),
50) 3-Oxoandrost-4-en-17β-yl 2′-chloro-4′-sulfamoylbenzoate (23),
51) 3-Oxoandrost-4-en-17β-yl 3′-carboxy-5′-sulfamoylbenzoate (24),
52) 3-Oxoandrost-4-en-17β-yl 3′-carbamido-5′-sulfamoylbenzoate (25).

10. Pharmaceutical compositions containing at least one compound according to.

11. Pharmaceutical composition according to claim 10, wherein at least one additional steroidally active compound is contained.

12. Pharmaceutical composition according to claim 11, wherein the additional steroidally active compound is a glucocorticoid, a gestagen or a GnRH analog.

13. Use of the compounds according to claim 1 for the production of pharmaceutical agents for hormone replacement therapy/substitution therapy in men and women.

14. Use of the compounds according to claim 1 of the production of pharmaceutical agents for birth control in men and women.

15. Use of the compounds according to claim 1 for the production of pharmaceutical agents for treating hormonally induced diseases in men and women.

16. Use of the compounds according to claim 1 for the production of pharmaceutical agents for treating diseases that can be positively influenced by the inhibition of the carboanhydrase activity.

17. Process for the production of compounds of general formula (I) according to claim 1 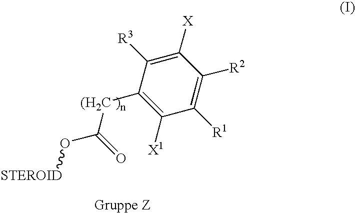
by reaction of androgens with sulfamoylphenylcarboxylic acid or derivatives thereof or by reaction of corresponding compounds with sulfamide, sulfamoyl chloride or aminosulfonyl isocyanate.
